(19) United States
(12) Patent Application Publication
(10) Pub. No.: US 2023/0008768 A1
(43) Pub. Date: Jan. 12, 2023

(54) DEVICE TO DIRECT URINE STREAM

(71) Applicant: Eric LEE, Laguna Niguel, CA (US)

(72) Inventor: Eric LEE, Laguna Niguel, CA (US)

(21) Appl. No.: 17/862,103

(22) Filed: Jul. 11, 2022

Related U.S. Application Data
(60) Provisional application No. 63/220,094, filed on Jul. 9, 2021.

(51) Int. Cl. A61F 5/453; A61F 5/44

(52) U.S. Cl. CPC A61F 5/453; A61F 5/4404; A61F 2005/4402

(57) ABSTRACT

A device to direct a urine stream has a circular collapsing sheath with a plurality of collapsible circular components. The circular collapsing sheath has a distal opening and a proximal opening. Each one of the plurality of collapsible circular components has a diameter smaller than the preceding one and the collapsing sheath collapses to a flattened form when subjected to a compressing force. The circular collapsing sheath opens to an expanded form when subjected to an expanding force. The circular collapsing sheath defines a continuous membrane that forms a channel that directs the urine stream into a waste receptacle. The circular collapsing sheath is made from a water soluble material that allows the user to discard the used device. The device is placed in a sanitary sealed package with a tear strip enabling the user to remove and use the device.

### CROSS REFERENCE TO RELATED APPLICATIONS

This application claims priority and herein incorporates by reference U.S. provisional patent application 63/220,094, filed Jul. 9, 2021.

### BACKGROUND OF THE INVENTION

Although there aren't many studies relating to urinating while standing, clearly many men in the United States urinate while standing up. There was a study in 2007 that indicated that 42 percent of married man urinate while sitting presumably to please their spouse. There are studies that show that the closer to the urinal or toilet the man is, the less splatter since urine flows tends to disperse into droplets and this leads to more splashing. An additional problem occurs when the penis is in an erect or semi-erect state, which can lead to problems in correctly directing the urine flow to the waste receptacle.

Additionally, as men age, a condition known as split urine stream is relatively common which causes inconvenience and embarrassment to the person suffering from this condition. This condition is often accompanied by dribbling which leads to problems and cleanup issues. Some new studies seem to indicate that as people become more concerned with the spread of infections such as viruses, urine splash is more of a concern.

There is a need for a simple, easy to use and sanitary device that solves the issue of urine direction, spills, and splashing.

### SUMMARY OF THE INVENTION

A device to direct a urine stream has a circular collapsing sheath with a plurality of collapsible circular components. The circular collapsing sheath has a distal opening and a proximal opening. Each one of the plurality of collapsible circular components has a diameter smaller than the preceding one and the collapsing sheath collapses to a flattened form when subjected to a compressing force. The circular collapsing sheath opens to an expanded form when subjected to an expanding force. The circular collapsing sheath defines a continuous membrane that forms a channel that directs the urine stream into a waste receptacle. The circular collapsing sheath is made from a water soluble material that allows the user to discard the used device. The device is placed in a sanitary sealed package with a tear strip enabling the user to remove and use the device.

Other features and advantages of the instant invention will become apparent from the following description of the invention which refers to the accompanying drawings.

### BRIEF DESCRIPTION OF THE DRAWINGS

FIG. 1 is an illustration of a device to direct urine stream according to an embodiment of the invention.

FIG. 2 is an illustration of the device to direct urine stream shown in FIG. 1 in use.

FIG. 3 is an illustration of the device shown in FIG. 1 directing urine stream towards a toilet.

FIG. 4 is an illustration of a device to direct urine stream according to an embodiment of the invention.

FIG. 5 is an illustration of a collapsible device to direct urine stream according to an embodiment of the invention.

FIG. 6 is an illustration of the device shown in FIG. 5 in an extended position.

FIG. 7 is an illustration of another collapsible device to direct urine stream according to an embodiment of the invention.

FIG. 8 is an illustration of the device shown in FIG. 7 shown in an extended position.

FIG. 9 is an illustration of another collapsible device to direct urine stream according to an embodiment of the invention that collapses to a very small profile.

FIG. 10 is an illustration of the device shown in FIG. 9 in an extended position.

FIG. 11 is an illustration of the device shown in FIG. 1 directing urine stream towards a urinal.

FIG. 12 is an illustration of a collapsing, packaging and opening embodiment of the device shown in FIG. 1.

FIG. 13 is a front view of the device shown in FIG. 9.

FIG. 14 is an illustration of the device shown in FIG. 9 enclosed in a sanitary package.

### DETAILED DESCRIPTION OF THE INVENTION

In the following detailed description of the invention, reference is made to the drawings in which reference numerals refer to like elements, and which are intended to show by way of illustration specific embodiments in which the invention may be practiced. It is understood that other embodiments may be utilized and that structural changes may be made without departing from the scope and spirit of the invention.

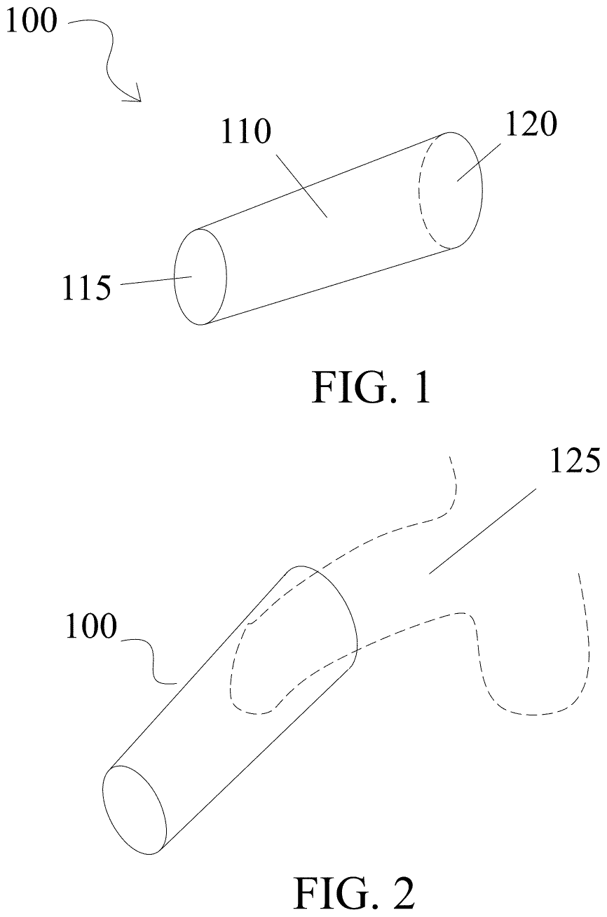

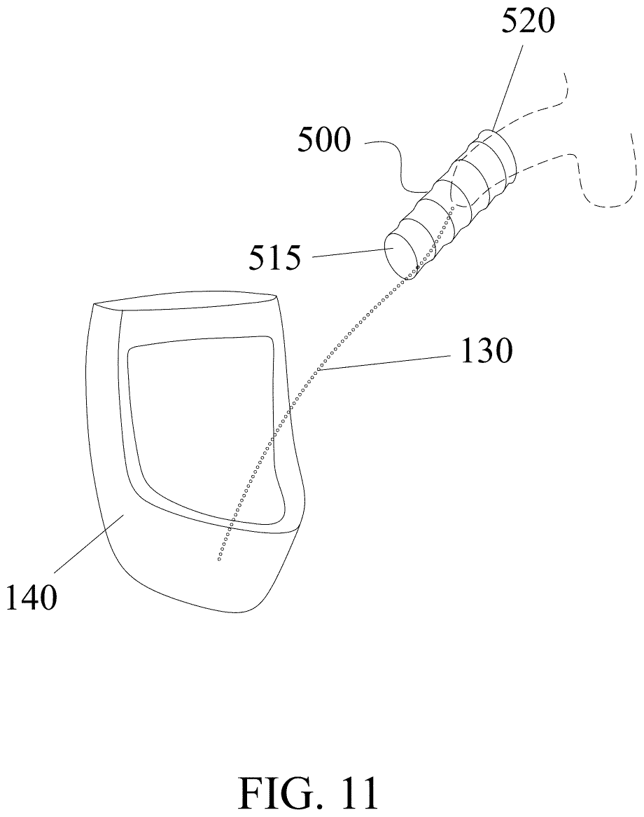

Referring to FIGS. 1-3 and 11, a device to direct urine stream 100 is shown having a sheath 110 which is generally tubular having a distal opening 115 and a proximal opening 120. In operation, a user's penis 125 is placed in proximal opening 125. This directs a urine stream 130 towards a toilet 135 or a urinal 140. In this way, urine stream 130 is both confined, directed and brought closer to toilet 135 or urinal 140 which helps reduce or prevent splashing. Additionally, any split urine steam will be redirected towards toilet 135 or urinal 140 thus preventing the issues normally accompanying this condition.

After use, device to direct urine stream 100 is disposed of by discarding in toilet 135 or urinal 140. The material making up sheath 110 is made of a material that dissolves in water such as Polyvinyl Alcohol (PVA), PVA coated paper or other water dissolving material. Of course other materials that do not dissolve in water may be used, such as but not limited to, silicone, plastic coated paper, etc., but if these materials are used, sheath 110 would need to be disposed of in a waste container (not shown) rather than simply disposed of in toilet 135 or urinal 140.

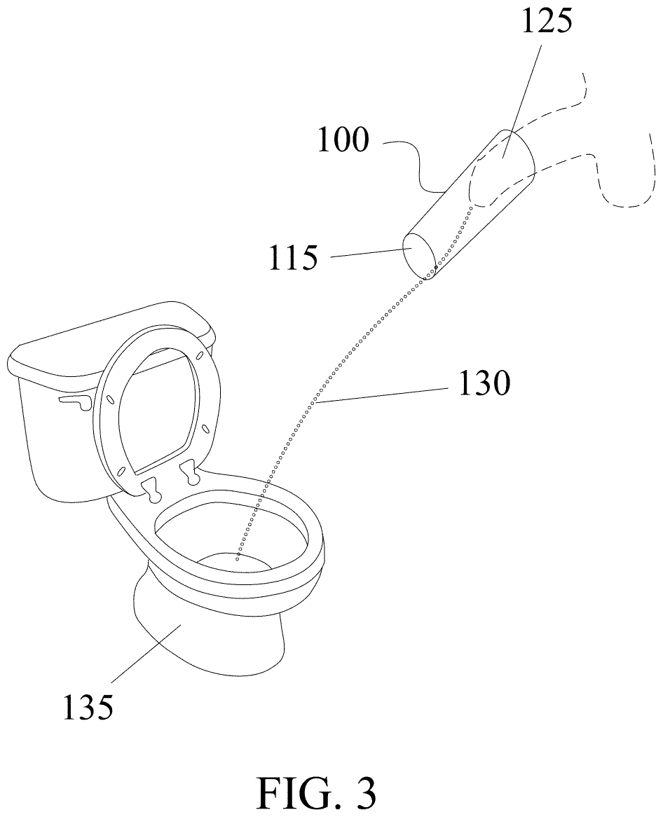

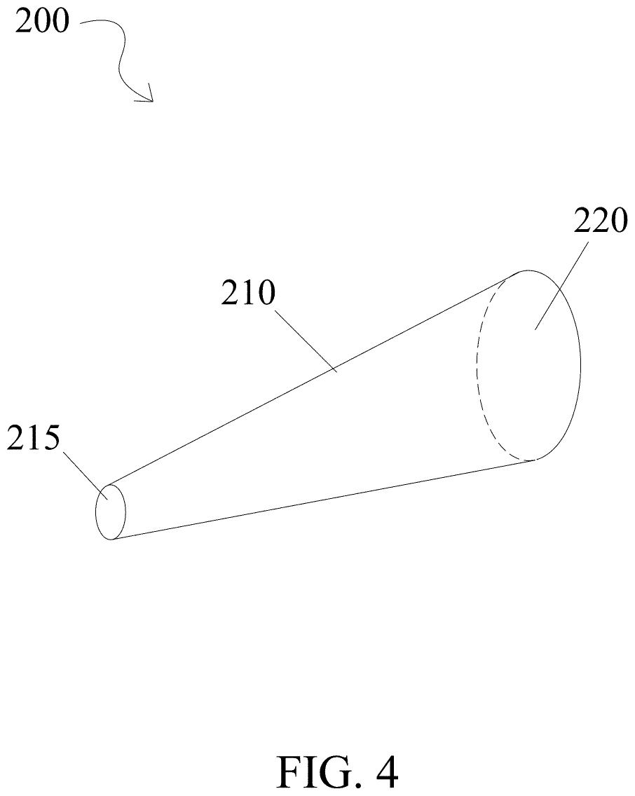

Referring now to FIG. 4, a device to direct urine stream 200 is shown having a highly tapered sheath 210 with a distal opening 215 and a proximal opening 220. As discussed above, the user places penis 125 (FIG. 2) in proximal opening 220, directing urine stream 125 (FIG. 3) towards toilet 135 (FIG. 3) or urinal 140 (FIG. 3).

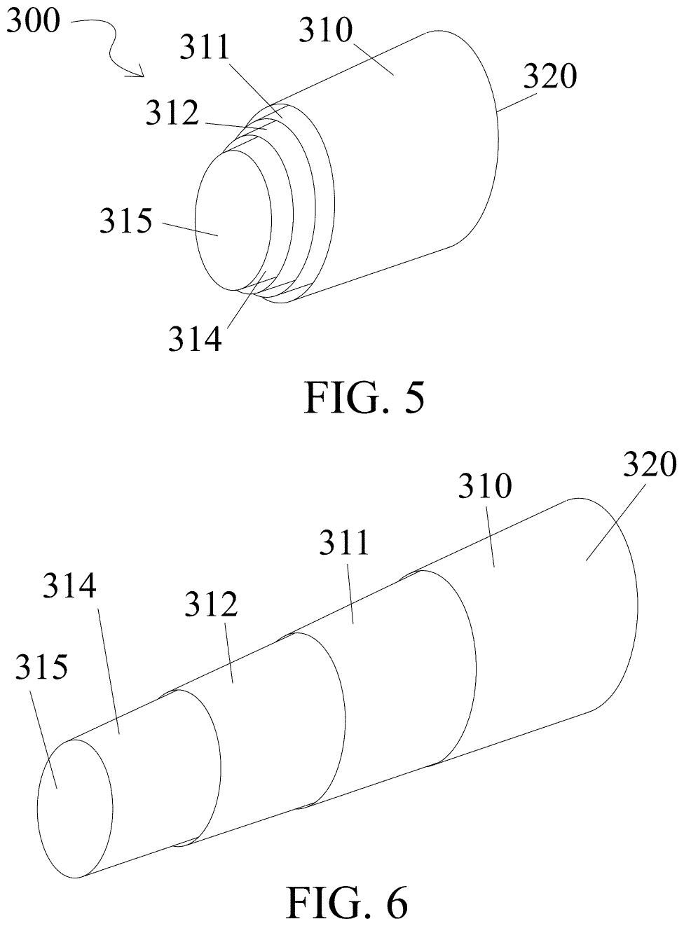

Now referring to FIGS. 5 and 6, a device to direct urine stream 300 is shown in a compressed form (FIG. 5) and an expanded .form (FIG. 6). Device to direct urine stream 300 has a telescopic outer sheath 310, a first telescopic inner sheath 311, a second telescopic inner sheath 312, and a third telescopic inner sheath 314. In operation, third inner sheath 314 is pulled out which expands device 300 to an expanded position and is used as described above.

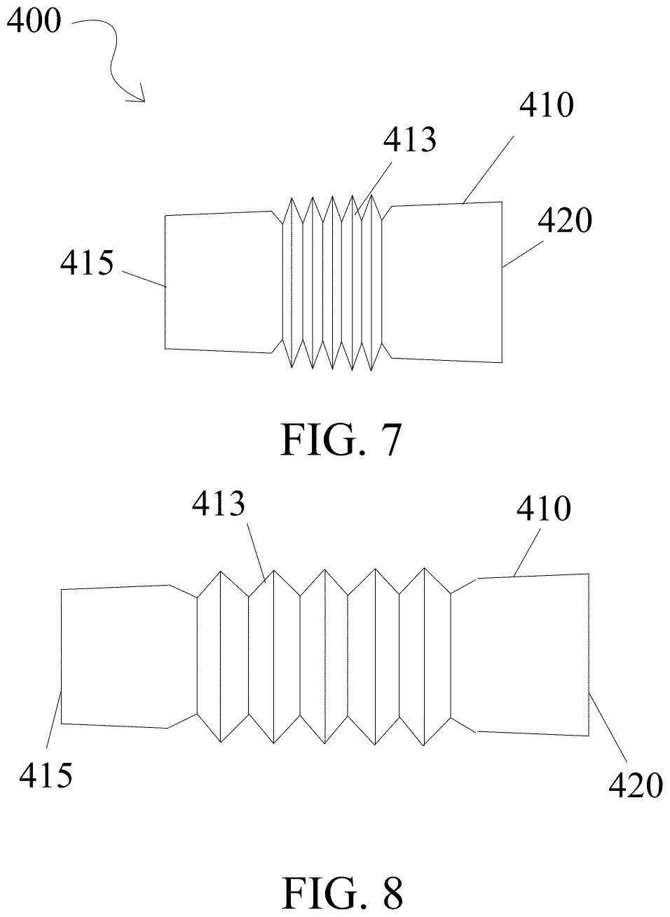

Referring to FIGS. 7 and 8, a device to direct urine stream 400 is shown having a sheath 410 and an expanding sheath portion 413 centrally located on sheath 410. Again, a proximal opening 420 and a distal opening 415 is shown and operates as described above. In operation the user pulls on expanding sheath portion 413 to an expanded position for use.

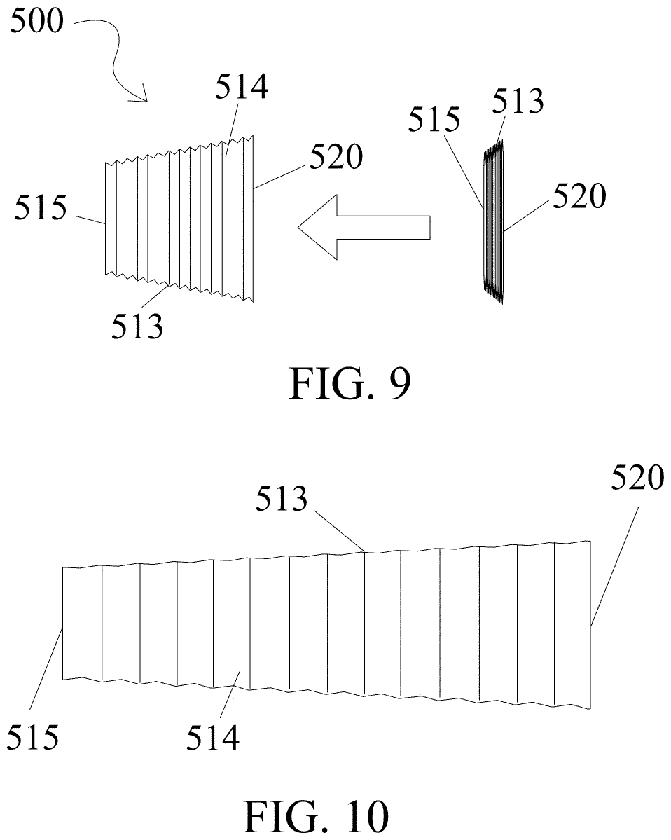

Now referring to FIGS. 9 and 10, a device to direct urine stream 500 is shown having a plurality of circular collapsible components 513 with a proximal opening 520 and a distal opening 515. Each one of the plurality of circular collapsible components 513 has a diameter smaller than the preceding one ranging from largest proximal opening 520 to smallest distal opening 515. In this way, the plurality of circular collapsible components form a continuous membrane 514 disposed between proximal opening 520 and distal opening 515 and keeps urine contained within. Said circular corrugated components are folds in continuous membrane 514. Of course circular collapsible components may be separate components such as wire, plastic or other suitable material as is known in the art as long as a leak proof membrane is achieved in order to completely contain the urine. in In this embodiment, a collapsed orientation is shown having a very small profile which allows device 500 to be stored in a small flat sanitary package (530 FIG. 14). In operation, device 500 is stretched into an expanded position for use. The actual number of collapsible components is not critical as long as device 500 can move between a collapsed orientation and a useful expanded orientation.

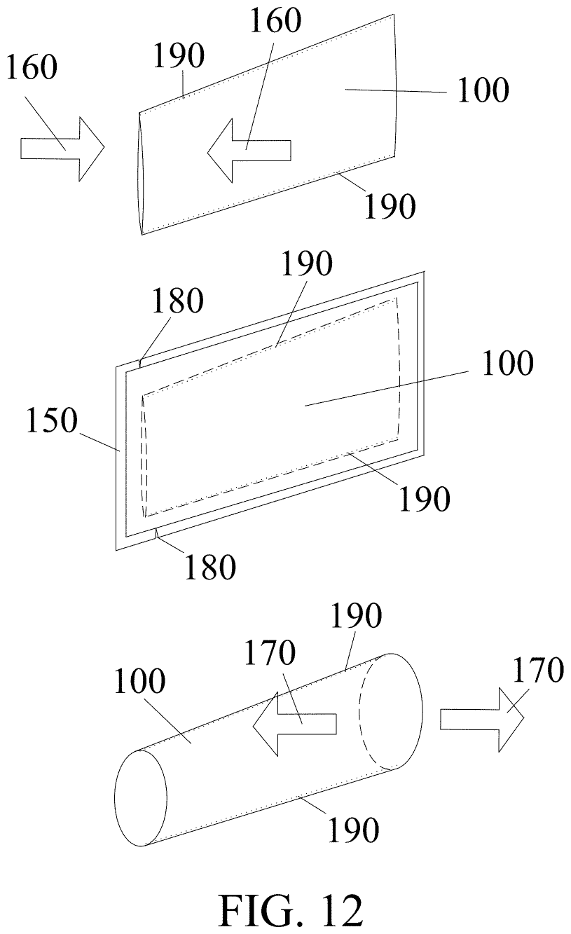

Referring now to FIG. 12, device to direct urine stream 100 is shown using a collapse action 160 to allow a generally flat package 150 and then using an opening action 170 to prepare for use. Device to direct urine stream 100 may include folding seams 190 to facilitate packaging and opening for use. Package 150 is a plasticized material with a tear strip 180 as is known in the art. This allows for sterilized hygienic packaging for convenience and portability. Of course other packaging means and materials may be used for all the embodiments shown in the figures including, but not limited to, bulk packaging, refillable container, or a pop-up container that allows a user to dispense one device at a time, or other suitable packaging and/or dispensing embodiment.

Again, in all embodiments, the material is made of a water soluble material to allow the user to simply dispose of the used device by discarding in toilet or urinal as discussed above. Of course other suitable materials may be used without departing from the inventive enablement of the instant invention.

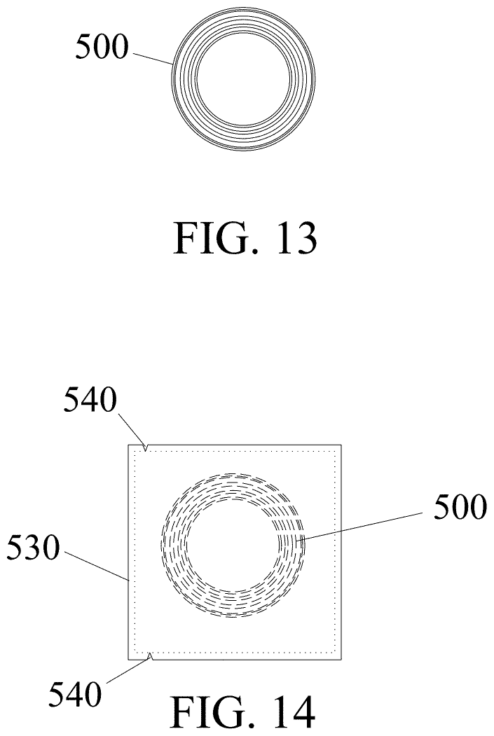

Referring now to FIGS. 13 and 14, device to direct urine stream 500 is shown collapsed and contained in a sanitary package 530 with tear strips 540 as is known in the art. The user can easily and sanitarily carry device 500 in a pocket and then simply tear open the package when needed.

Although the instant invention has been described in relation to particular embodiments thereof, many other variations and modifications and other uses will become apparent to those skilled in the art.

What is claimed is:

1. A device to direct a urine stream comprising:
a circular sheath;
said circular sheath having a distal opening and a proximal opening; wherein said distal opening has a smaller radius than said proximal opening;
said proximal opening having a diameter adapted to fit a user's penis; and
said circular sheath adapted to direct said urine stream into a waste receptacle.

2. The device according to claim 1 wherein said circular sheath is made of a water soluble material.

3. The device according to claim 2 wherein said water soluble material is Polyvinyl Alcohol.

4. The device according to claim 1 further comprising a first folding seam opposite to a second folding seam wherein said device is collapsible and expandable along said folding seams.

5. The device according to claim 4 further comprising a sterilized package to removably contain said device.

6. The device according to claim 1 wherein said distal opening is at least half the diameter of said proximal opening.

7. A device to direct a urine stream comprising:
a circular collapsing sheath;
said circular collapsing sheath having a plurality of collapsible circular components;
said circular collapsing sheath having a distal opening and a proximal opening;
each one of said plurality of collapsible circular components having a diameter smaller than the preceding one; wherein said collapsing sheath collapses to a flattened form when subjected to a compressing force;
said circular collapsing sheath opening to an expanded form when subjected to an expanding force; and
said circular collapsing sheath defining a continuous membrane forming a channel adapted to direct said urine stream into a waste receptacle.

8. The device according to claim 7 further comprising a sterilized package to removably contain said device.

9. The claim according to claim 7 wherein said circular collapsing sheath is made of a water soluble material.

10. The device according to claim 9 wherein said water soluble material is Polyvinyl Alcohol.
